(19) United States
(12) Patent Application Publication
(10) Pub. No.: US 2009/0112078 A1
(43) Pub. Date: Apr. 30, 2009

(54) Embeded advanced force responsive detection platform for monitoring onfield logistics to physiological change

(76) Inventor: Joseph Akwo Tabe, Silver Spring, MD (US)

Correspondence Address: Joseph A. Tabe, Suite 717, 11700 Old Columbia Pike, Silver Spring, MD, 20904, US

(21) Appl. No.: 11/977,365

(22) Filed: Oct. 24, 2007

(51) Int. Cl. A61B 5/04; G06F 15/00

(52) U.S. Cl. 600/388; 702/189

(57) ABSTRACT

A wearable outfit comprising nano-sensors for continuous physiological condition and body temperature monitoring configured with load cells and strain gages for monitoring and measuring the degree of force impacted on an athlete during a hit or collision in a sporting event such as a football game, the strain gages are composed of electrical resistance elements embedded in a micro-fibered material and etched in a silicon substrate and located in a position that is in contact with the body of the wearer. The wearable outfit also enables wireless communication to computer device configured for algorithm and analysis of the detected conditions and for signaling personnel about the severity of an injury or collision through either auditory or visual device. The computer device is configured with algorithms for enabling accurate interpretation of the physiological condition or body surface temperatures of personnel.

APPLICANT HEREBY CLAIMS PRIORITY BENEFITS UNDER 35 USC 119 OF THE PROVISIOANAL APPLICATION. Ser. No. 60/426,800. Filing Date Nov. 18, 2002, Ser. No. 10/660,473, Filed Sep. 12, 2003, now U.S. Pat. No. 7,271,720, Ser. No. 11/821,776. Filed Jun. 25, 2007.

### FIELD OF THE INVENTION

This invention relates to the field of sporting events comprising nano-sensors for monitoring continuous physiological conditions and body temperature and collision force measuring wearable outfits, specifically to a wearable outfit that can monitor the physiological conditions, body temperature, and exacted collision force derived from at least an incidental collision with a person in a sporting event. The persons could be athletes and playing at least a football game, or riding a bicycle.

### BACKGROUND OF THE INVENTION

In a sporting event that constantly athletes are subjected to routing hits, body parts under-go severe stresses and strains. Regularly, these athletes sustain severe injuries that are live threatening and some times paralyzed. The athletes, in fear of their future some times continue playing without the slightest idea about the severity of the injuries. Additionally, some events like football has become so physically developed that monitoring the physiological condition of players during a game is eminent. Communicating any detection to the sideline wireless to a computer device will expedite the safety and security of these players. Regularly players are reminded of the dangers imposed from exercise and/or playing in severe environmental conditions Such as hot and/or cold weather. Though well conditioned athletes and military recruits sometimes are afflicted with heat illnesses and deaths with predictable regularity, still the incidence of high profile deaths and heat related deaths continue to occur. Therefore, without the use of a wearable outfit that comprises nano-sensors for continuous physiological condition and body temperature monitoring, and configured with load cells and strain gages for monitoring the degree of force impacted on an athlete during a hit or collision in a sporting event such as a football games, any given game day a player is susceptible to the illnesses the game is subjected to.

Thought children athletes are more susceptible to heat related illness due to their higher metabolic rates and body composition, they have diminished capacity for sweating and their bodies need to be monitored for heat rise since occasionally they may not hydrate themselves properly. The monitoring of physiological conditions, impacted force, and body temperature are very important in ensuring that the players health are promptly reported at the sideline to prevent any further exposure to elevated injuries. The conditions that exist which cause a dangerous elevation in a person's physiological condition and body temperature are detectable and communicated to prevent any physical response occurring that can be harmful and sometimes fatal to the injured person, including dizziness, fainting and cardiac arrest.

The wearable outfit is a revolutionary multipurpose nanotechnology application through a detection platform to enable collision force measurement, detection, protection, and monitoring of and intervention into sporting environments. The device consist of nano-sensors embedded in silicon substrate and etched/fused in a micro-fibered material having excellent electrical characteristics to enable effective and efficient detection platform responsive for detection of vast common collision and physiological conditions in response to various emergency conditions in a sporting event. The device comprises a computer device configured for responses to the analytical detection data. The device focuses on sensitivity and selectivity of current and projected forms of common emergency associated with the nature of various sporting events for enabling detection of and protection against dangerously extended injuries/conditions through monitoring, protecting and communicating during sensitive and selective sporting environments. The outfit further protects the body against body bacteria from environmental conditions, and monitors personnel physiological signs, their heart rates, and their respiratory system, enabling the computer device to report all data and detected information to the sideline. The wearable outfit is interactively configured with the computer device to enable instant response to anticipatory physiological conditions.

The invention comprises nanotechnology based outfit for enabling collision force measurement, detection and communication, a revolutionary multipurpose application through a detection platform configured to enable detection, protection, and monitoring of personnel physiological conditions in a sporting environment such as a football game. The outfit consists of nano-sensors embedded in silicon substrate and etched/fused in a micro-fibered material having excellent electrical characteristics to enable effective and efficient detection platform responsive for monitoring the physiological conditions such as heart rate, vital signs, and blood pressure.

### SUMMARY OF THE INVENTION

The preferred embodiment of the present invention is a wearable outfit configured with nanotechnology application to enable continuous measurement of impacted force sustainable in a sporting event, such as at least a football game, whereby the exacted force is measured and the physiological condition of at least one of the players in the sporting event is monitored, and whereby these force measurements and the physiological detections of the players conditions are communicated wirelessly to at least a computer device positioned at the sideline.

The wearable outfit comprised of nanotechnology consisting of embedded nano-sensors such as MEMS and other force measurement sensors such as load cells configured with strain gages, at least one of the nano-sensors is configured so that its resistance changes with at least a sensed force, at least a physiological change, including body temperature, heart rate and high blood conditions. The sensors are configured with miniaturized antennas and miniaturized sensors, and communicatively connected wirelessly to a computer device to enable at least a readout of any detection to the medical staffs, the coaches and/or personnel on the sideline so that an early warning of the athlete's situation and/or condition is enabled to prevent any further dangerously alarming condition.

The wearable outfit comprises at least micro-fibered and/or silicon substrate having embedded miniaturized antennas, miniaturized sensors, and at least battery powered. The wearable outfit further contains an RFID chip configured for enabling communications to the sideline and can be programmed to trigger an audible signal, such as an audible beep, or visual signal, such as the readout when at least detection is enabled. The computer device eying these biological/force measurement sensors is an analytical tool that consists of biologically active materials such as surface resonance spectroscope and is used with devices that will convert biochemical signal into quantifiable electrical signal to enable communication of all detected information through the electrical signals or pulses traveling between the detection platform and the computer device. These signals are transported wirelessly through waves such as radio waves or microwaves to the sidelines.

Prior devices have failed to address measurement of collision forces in physical sports such as a football game and have no way of extending their sensitivity to detecting physiological conditions of players during the event. With the present invention, the area of force measurements, protective sensing, and physiological conditions of personnel is not limited to the analytical techniques of detecting and biometrics but rather extends beyond saving lives and reducing the risk of dangerously extending an injury.

The advancement of the wearable detection outfit comprises force measurement sensors and biological sensing elements which would measure collision force during a common collision in a sporting event and selectively recognize a particular biological molecule through a reaction specific body adsorption, or other physical or biochemical processes, allowing the sensors to convert the result of its recognition into a usable signals which are quantifiable and amplifiable. Typical sensors for this invention further include optical, electro-optical, or electro-biochemical devices to enable manly sensing environment on the platform for specific applications to translate physical or biochemical change.

### BRIEF DESCRIPTION OF THE DRAWINGS

FIG. 1 depicts a detection platform showing of nanotechnology application on a material for an outfit.

FIG. 2 depicts the detection platform fused/etched in a second material to enable the outfit.

FIG. 3 depicts a first designed outfit comprising nanotechnology applications.

FIG. 4 depicts a configuration of the nanotechnology application for football applications.

FIG. 5 depicts the outfit worn by a football player and sensing body physiological change.

FIG. 6 depicts the outfit to be worn by a player for monitoring various body conditions associated with the physical nature of a football game.

FIG. 7 depicts sections of the detection platform having means to absorb force energy upon collision.

FIG. 8 further depicts the detection platform con figuration to absorb and distribute the force energy.

FIG. 9 is seen to represent a football quarterback player completely outfitted with the embedded outfit.

FIG. 10 is seen to represent a football wide receiver wearing the outfit and awaiting to catch the football released by the quarterback.

FIG. 11 is seen to represent a football safety wearing said outfit and awaiting to level the receiver.

FIG. 12 is seen to represent the football released by the quarterback to the direction of the receiver.

FIG. 13 is seen both the receiver and the safety wearing said outfit and in hard collision.

FIG. 14 depicts team personnel observing the report of the collision severity.

FIG. 15 depicts a computer system on the sideline showing the severity of the collision and reporting all on-field logistics to any physiological change.

FIG. 16 is seen a football coach querying the outfit for a report on any further health risk associated with the collision.

FIG. 17 depicts NFL network system tracking the on field body logistics through direct communication s with sideline computers.

### DETAILED DESCRIPTION OF AN ENABLING AND PREFERRED EMBODIMENT

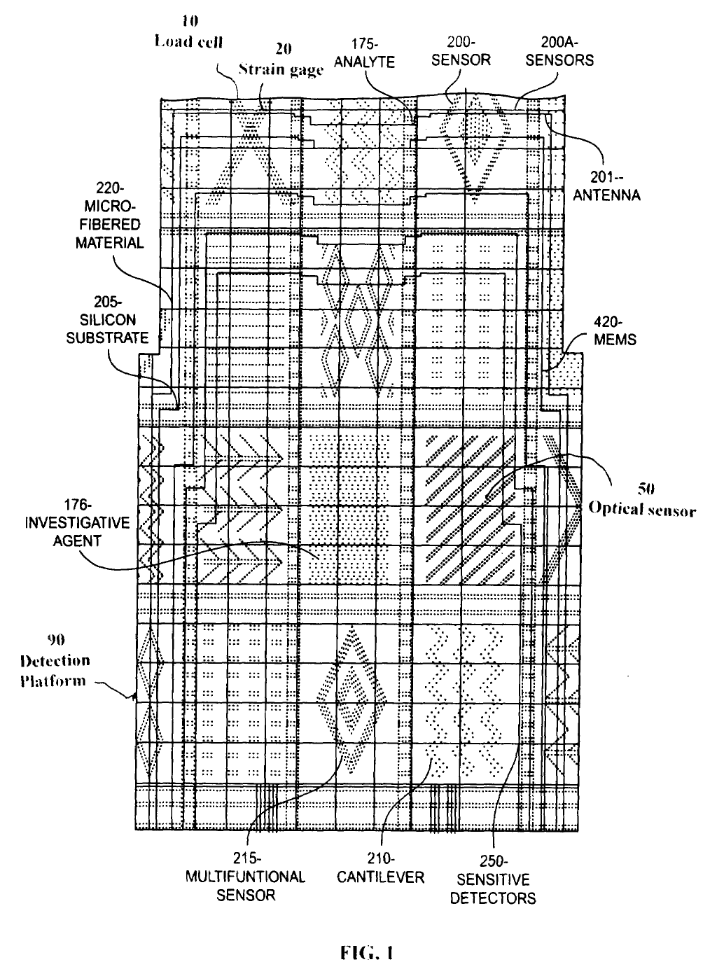

The present invention consist of nanotechnology applications as seen in FIG. 1, including at least MEMS 420. RFID CHIP 200, load cells 10, strain gage 20, sensors 200A comprise optical sensors 50, electro optical sensors 60 and may include other nano-sensors such as cantilever sensor 210, multifunctional sensors 215, and at least a piezoelectric sensor all embedded in a silicon substrate 205 and fused in a micro-fibered material 220 to enable a detection platform 90 that enable communications. The detection platform 90 comprises the micro-fibered material 220 etched on at least a second material to comprise a wearable outfit 100 for athlete's security applications, such as on-field body logistics to physiological change in at least football applications.

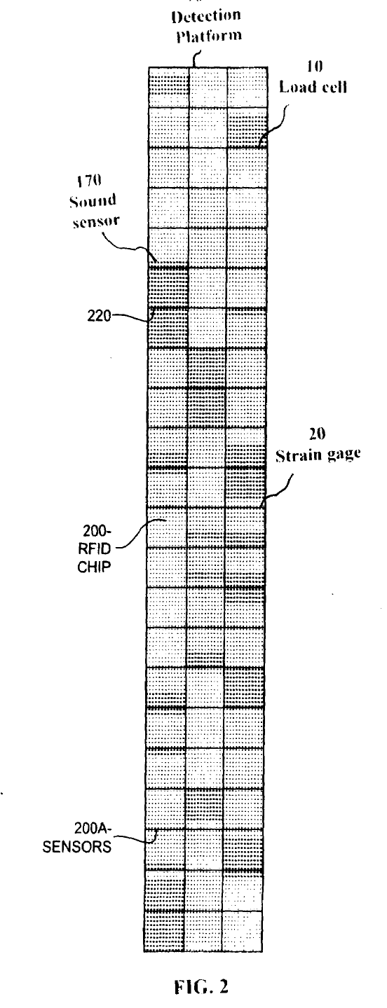

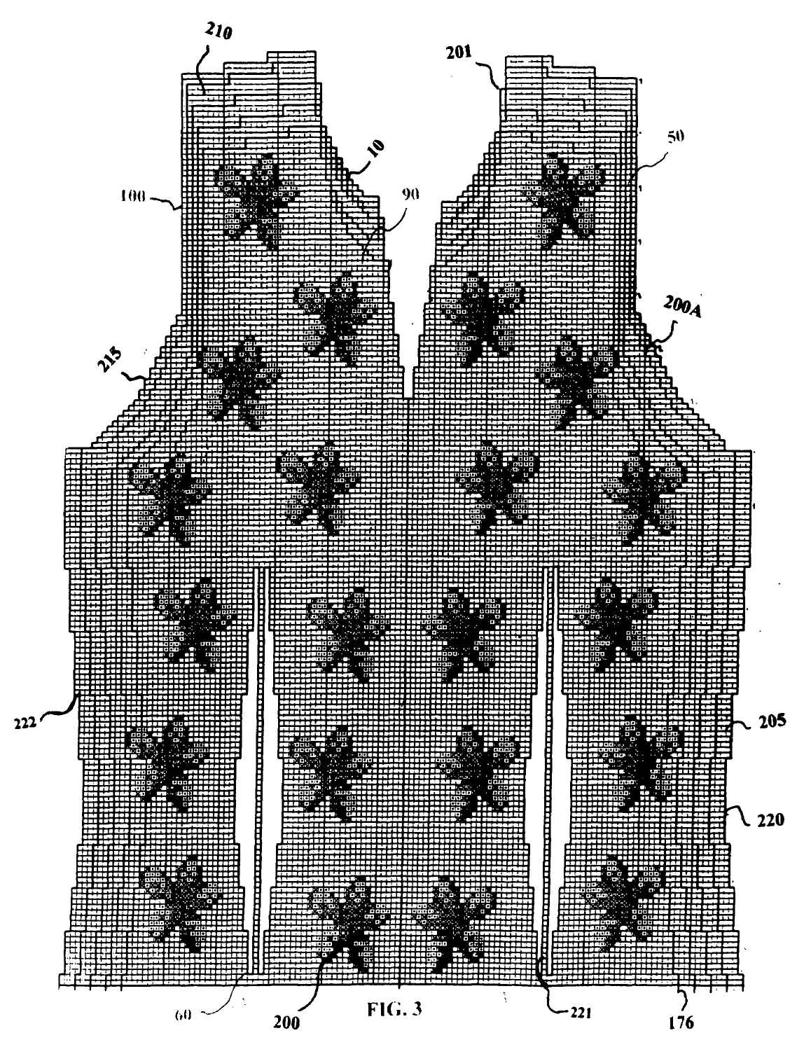

FIG. 2 further depicts the nanotechnology application for a detection platform 10 comprising Sound sensor 170 operatively configured with an RFID chip 200. FIG. 3 depicts the detection platform 90, which has been transformed into a force responsive outfit 100 for on-field body logistics to detecting physiological change.

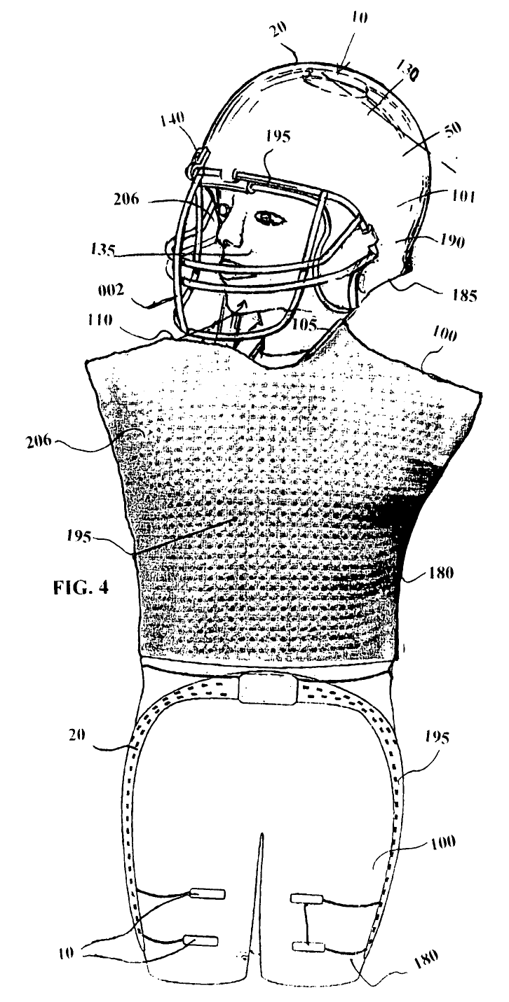

FIG. 4 depicts the present invention outfitting a player 110 with the outfit 100, which comprises at least a helmet 101 which has the detection platform 90 which has been embedded with load cells 10 and strain gage 20. The detection platform may include at least a first detection means 190, and may include at least a back wall 140, an upper wall 130, an optical sensor 50, a side walls 135, a membrane 195, a resilient membrane 206, an interior wall surface 225, a transducer 185, and a second detection means 180.

FIG. 5 depicts the present invention showing nanotechnology applications on an outfit 100, further depicted to sense applied force on the body 105 of a player 110, such that the detection platform 90, upon sensing a force, monitors the physiological conditions of the player 110 who absorbed the force on the body 105. The force is first absorbed by the silicon substrate 205 or a load cell 10, and distributed within the detection platform 90, so that the actual force exacted on the body 105 is reduced. The strain gage transforms the force exacted upon the body 105 of a player into measurable force which is then communicated to the sideline, including any physiological change within the body after the hit.

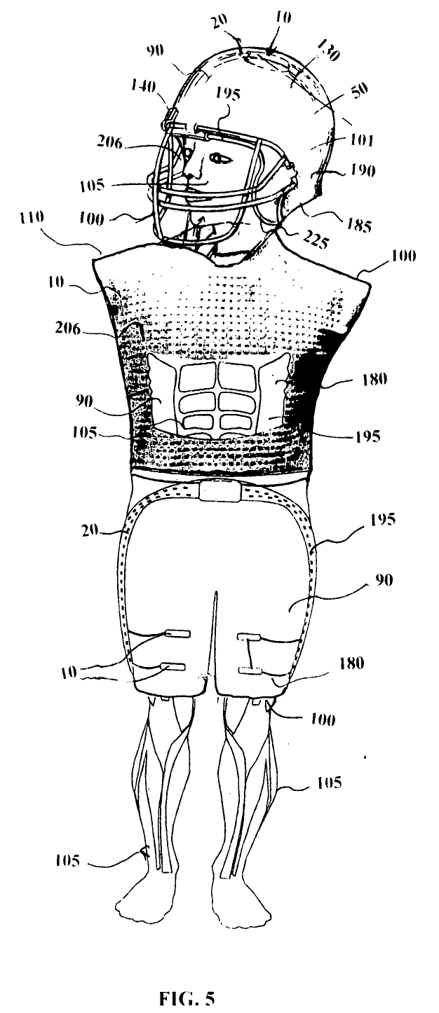

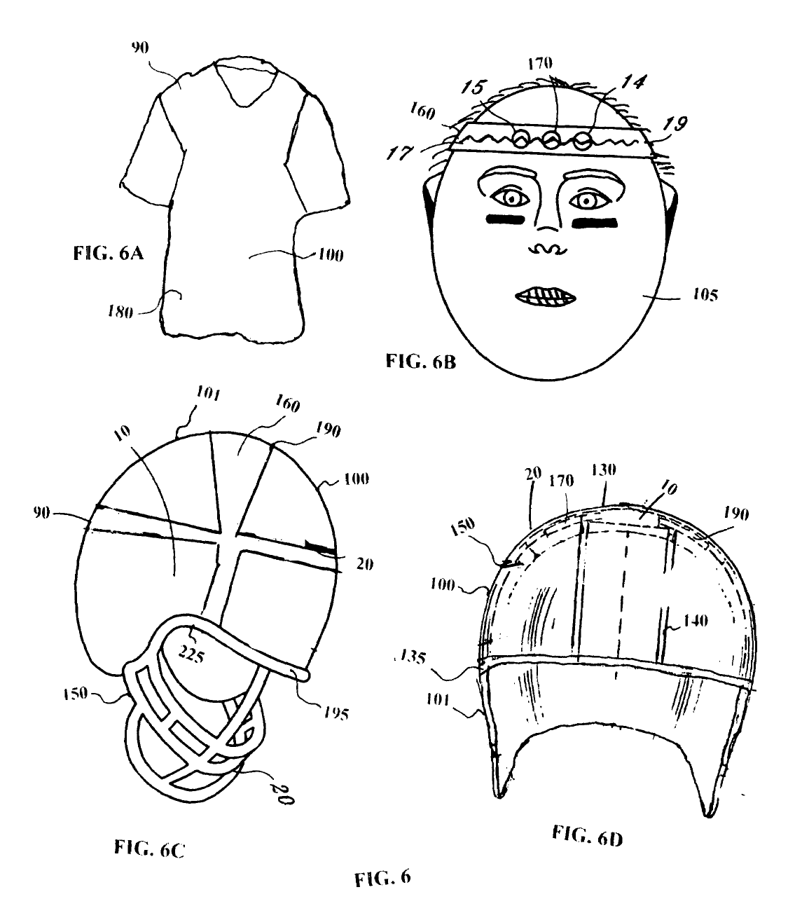

FIG. 6a depicts the outfit 100 configured with the detection platform 90. FIG. 6b depicts the body 105 of a player which is likely to sustain major injury after a severe hit, which may subject the body 105 experience a headache 19, extended fever 17, increased brain pressure 15, or a blackout 14. FIG. 6c depicts the present invention showing the operative configuration of the helmet 101 that is required to enable maximum protection against the various likelihood common injuries. The helmet comprise the various configuration of the present invention, including a membrane 195, a shell 150, a load cell 10, a strain gage 20, an electro-optical sensor 160, and a first detection platform 190. FIG. 6d depicts the rear of the helmet 101, showing extensions of the protection.

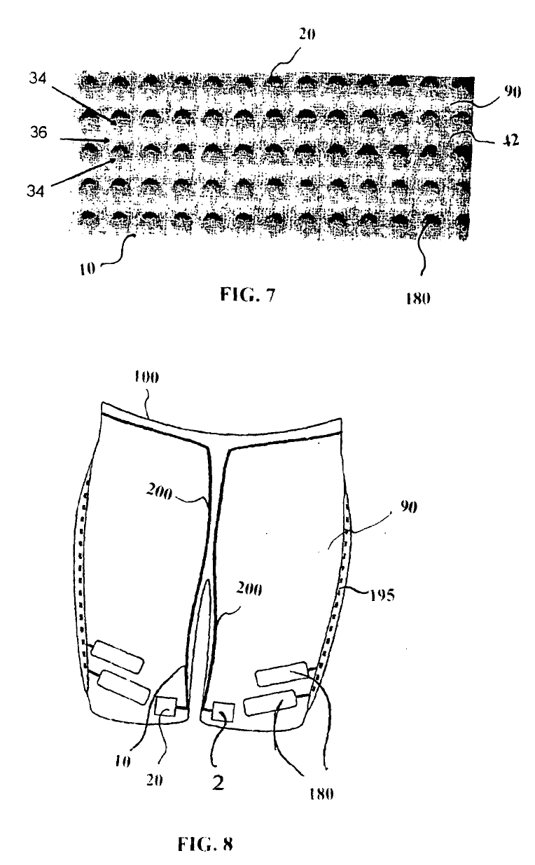

FIG. 7 depicts the portion of the detection platform 90 that is configured with means to absorb force and distributing this force ports 34, 36, and 42, so that the actual penetrative force into the body 105 of player 110 as shown in FIG. 4, is greatly reduced. FIG. 8 depicts the detection platform 90 which has been transformed into a lower body outfit 100, and configured with means to sense the likelihood of body tear which is being monitored by a piezoelectric sensor 2.

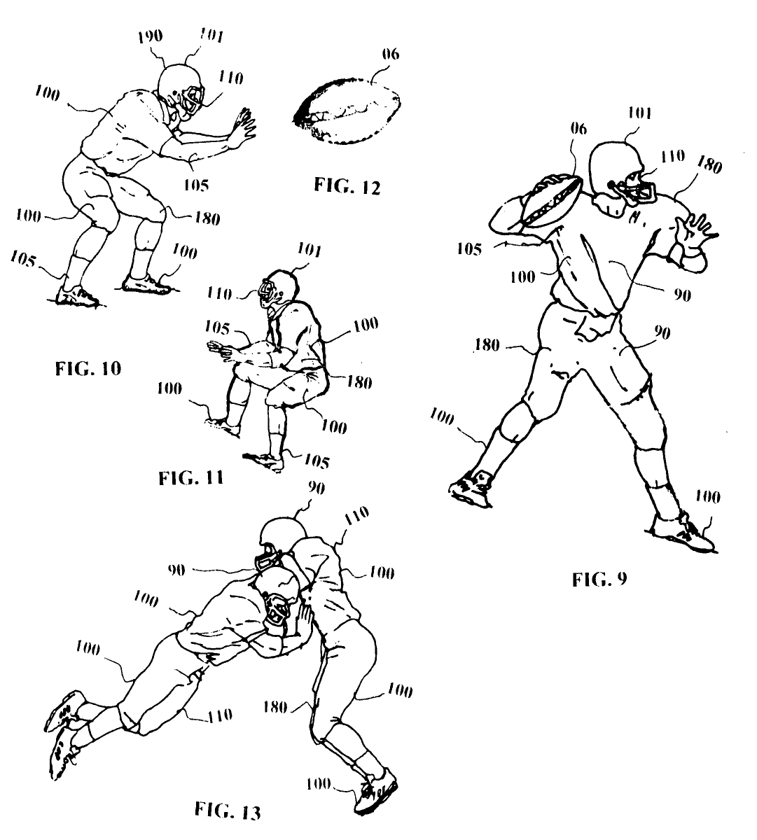

Referring to FIG. 9 is seen a quarter back 110 outfitted with the outfit 100, comprising a detection platform 90, a second detection means 180, and a helmet 101. The quarterback 110 is seen throwing the ball 06 to a receiver shown in FIG. 10. Referring to FIG. 11 is seen a safety 10 wearing the outfit of the present invention and positioned to release a hit on the receiver 110 of FIG. 10. Referring to FIG. 12 is shown the football 06, which has been released by the quarterback. Referring to FIG. 13 is shown the safety releasing a severe hit on the receiver, and the outfit absorbing the hit and distributing the force away from the receiver's body, while also monitoring any bodily damage or injury that is likely sustained from such hit.

The load cells 10 is configured to sense applied force on the body of at least a player 110 and the strain gage 20 is responsive for transforming the impacted force on the body of the player 110 into measurable electrical energy readable wirelessly by a computer device 400 located at the sideline. At least one of the plurality sensors is configured to read the strain gage 20 signals to enable a continuous monitoring of the player's physiological conditions. At least one of the plurality sensors further enables communications between the detection platform 90 and the computer device 400 through antennas 201.

The silicon substrate 205, micro-fiber material 80, and the other plurality nano-sensors require processes that are unique to advanced sensitivity and selectivity. Other embodiment of the inventive methods include ferrous 001 and/or nonferrous 002 material alloyed with the micro-fibered material 220 and embedded, fused, or etched to enable material toughness that would enable collision force absorption. The non-ferrous material 002 may comprise miniaturized materials, such as nano-particles of a non-ferrous material 221. Still, other embodiment of the inventive methods comprises malleable miniaturized steel in the alloying process to enable advanced toughness comprising more force absorption through the wearable outfit 100. Still in the inventive methods, focus is further concentrated on the elastic properties of the alloying materials to enable the wearable outfit 100 exhibits elastic shrinkage to support key injury prom areas like the joints and also enables collision force absorption. Still in the inventive method, the reinforcement consist of other material properties that include elasticity and/malleability for absorbing more of the collision force to be impacted on the player's body 105. In other embodiment of the invention, the methods further consist of alloying the miniaturized steel material with a micro-fiber material such as polypropylene in a silicon substrate 205 to enable re-enforcement of the outfit.

FIG. 13 further shows a view of the outfit 100 comprising a helmet 101 arrangement on top of the head of player 110. The helmet 101 comprises nano-sensors, load cells 10, strain gage 20, and other materials such as miniaturized steel. The detection platform 90 is communicatively connected to the nano-sensors. FIG. 5 is a schematic view of the entire wearable outfit 100. The wearable outfit 100 further encloses the detection platform 90 for measuring physiological conditions of the players 110, including body temperature and heart rate readings through the sensors configured to enable communications through antennas 201 configured with the plurality sensors to the computer device 400 at the side line as shown in FIG. 15. The wearable outfit 100 is operatively configured with the computer device 400, further responsive for producing an audio/visual signal when the body temperature reaches a critical threshold that will trigger unsafe condition for the player 110, which is to be relayed to the sideline personnel for prompt responses. The nano-sensors may further include piezoelectric sensors such as a piezoelectric transducer 185 for the wearable outfit 100 to produce mechanical motion of the body or force measurement in response to body electrical signal. The piezoelectric transducer 185 could also be programmable and used as a receiver in the present invention.

FIG. 6 is a detailed view of the helmet 101 which also comprise a physiological condition monitoring system. In the event a body part experiences broken sound, a sound sensor 170 would translate a sound wave at the instant of the snap to the computer device 400. The sensors are flexible and bonded to or embedded into the silicon substrate 205 operatively configured with a structural membranes 195 communicatively connected to a second material for the wearable outfit 100. The detection platform 90 is used to telemeter data as acoustic waves through the antenna. Proper spacing of the plurality sensors and phasing of the antenna for signal communication is directionally enhanced or encoded to improve transmission efficiency. The plurality sensors further enable monitoring physiological conditions of players 110, which further include health conditions.

Referring back to FIG. 7 further shows the detection platform 90 configured with the plurality sensors. The Load cell 10 is configured to measure force applied to a player 110 upon collision. A strain gage 20 is configured with the load cells 10 to transform the applied force into electrical energy. The strain gage 20 is further configured with sensors 200, which may include piezoelectric sensor for converting electrical energy into acoustic energy, and vice versa. Referring to FIG. 8 is seen the outfit 100 configured with the detection platform 90, the wedge membrane 195, the load cell 10, the strain gage 20, sensors 200, and piezoelectric sensor 2.

The thin flexible piezoelectric sensor 2 is used to input electrical energy to induce acoustic waves in the structural membrane 195 or receive electrical energy produced by acoustic waves in the structural membrane 195 of the wearable outfit 100. The piezoelectric sensor produces corresponding acoustic waves and the computer device 400 receives the acoustic waves and produce corresponding electrical signals containing the personnel's biometrics. In another embodiment, at least a sensor is etched/bonded to a surface of the structural membrane 195 of the wearable outfit 100 for health monitoring. In another embodiment, the sensors are etched/bonded at key contact points with the personnel's body 105 that are normally subjected to collision force, such as the head 160 and the chest area for monitoring structural change and physiological conditions of the player 110.

In other aspect of the invention, wearable outfit 100 comprises substantially elastic/non-elastic compressible/incompressible composition configured substantially not to quickly enable self-level deformation under standard operating conditions such as the physical nature of a football game. The detection platform 90 comprises a suspending agent which reacts substantially as a solid when subjected to forces above a normal force, and which exhibits some protection to a player 110 in a substantially injury based area through subjected forces above the normal force. The wearable outfit 100 further comprises a composition whereby provision is made for an incident energy absorption, such as a collision force released by a “termed” safety personnel in a football game in which the incident is normally seen wherein a quarterback throws a football to a mobile/immobile receiver. The incident generates a scene whereby the collision energy may be monitored as sound energy which may be communicated to the sideline, including all the properties that may be generated by the collision force and sound, such as body heat or increased heart rate. Still, the embodiment of the present invention further includes an object of enabling multiple absorption of a collision force in a football game through a wearable outfit 100 such as a protective helmet 101.

Another feature of certain embodiments of the present invention is that the at least one nano-sensor may comprise first and second detection means 190, 180, whereby the first detection means 190 being communicatively received within the second detection means 180 when a relative motion is sensed between the first and second detection means. An additional feature is that the detection platform 90 may be disposed with at least one nano-sensor and may include at least one wedge membrane 195 that is engage-able with an interior wall surface 225 of one of the detection means to substantially prevent relative motion impact between the first and second detection means 190, 180. A further feature is that the detection platform 90 may be associated with the first and second detection means 190, 180, and the second detection means 180 may have a plurality of antennas 201 formed in the interior wall surface 225 and operatively configured with at least one wedge membrane 195 communicatively engage-able with at least one of the plurality sensors.

Another feature of certain embodiments is that at least a sensor may be associated with the force sensor on the detection platform 90 comprising nanotechnology applications, and upon a predetermined force being sensed by the force sensor, activation of the detection platform 90 is enabled to cause at least one wedge membrane 195 to operatively engage the interior wall surface 225 of one of the detection means 190, 180 in wireless communication to the computer device 400. The nanotechnology application may include MEMS 420 in communication with the detection platform 90, or alternatively, may include at least a strain gage 20 configured for measuring collision force and for enabling electrical communication with the detection platform 90.

Further embodiment of the present invention comprises features Such as the first end of the detection platform 90 configured with at least one nano-sensor comprising connection assembly connecting the first end of at least one nano-sensor to one of the walls of the protective helmet 101, whereby the connection assembly further including at least a connector, whereby the first end of at least one nano-sensor may pivot with respect to the wall of the protective helmet 101.

Another aspect of the embodiments of the present invention consist of wearable outfit 100 comprising at least a protective helmet 101 having at least an upper wall 134, at least two side walls 135, and at least a back wall 136. The wearable outfit 100 may further include a force sensor disposed within the structural configuration of the walls of the protective helmet 101; at least one nano-sensor configured with first and second ends, the first end of the least one nano-sensor adapted to be associated with one of the walls of the protective helmet 101 and the second end of the at least one nano-sensor associated with enabling specific detection; the at least one nano-sensor enabling detection of physiological conditions of players 110 relative to sensed impacted force on the personnel body 105; and a detection platform 90 associated with the at least one nano-sensor, whereby the detection platform 90, upon a predetermined force being sensed by the force sensor, enables data communication to at least a remote computer device 400 located at the side line. The detection platform 90 of the present embodiments is associated with the first detection means 190 and the second detection means 180 and comprise of nanotechnology application comprising plurality of antennas 201 formed in the interior wall surface 225 of the detection means 190, 180.

The configuration of the protective helmet 101 with silicon substrate 205 and micro-fibered material 220 and alloying the associate material for the helmet 101 with ferrous 001 and/or non-ferrous 002 material when compared with previously proposed conventional helmet 101 offers unique protection against injuries caused by impact forces exerted upon the top of the protective helmet 101, such as, for example, during the playing of the game of football.

The helmet 101 is shown to generally include a shell 150 having an upper wall 134, two side walls 135, and back walls 136. A force sensor comprising at a load cell 10 is configured with a strain gage 20 and disposed at the walls comprising the shell 150; at least one nano-sensor is associated with one of the walls of the shell 150; and a detection platform 90 is associated with at least one a detection means comprising at least a nano-sensor. When a predetermined force is sensed by the force sensor, the detection platform 90 is enabled to classify the physiological condition of the players 110.

The shell 150 receives the head 160 of the players 110 wearing the helmet 101. In another embodiment, the shell 150 further comprises at least a detection means and a conventional shock absorbing characteristics associated with the detection means. Shock absorbing characteristics may comprise a detection platform 90 configured with plurality of resilient membrane 206 responsive for absorbing forces exerted upon the shell 150, and the plurality of resilient membrane 206 are disposed within the detection platform 90. When the amount of the predetermined force is sensed by the force sensor, the detection platform 90 is enabled.

The magnitude of the force which is sensed by the force sensor “load cell 10”, to enable the detection platform 90 may be varied as desired, which may include factors such as weight and age. Preferably, each nano-sensor is configured within a detection platform 90. FIG. 1 depicts a detection platform 90 configuration with a wedge membrane 195 are engaged with the interior wall surface 225 of at least a detection means comprising of nano-sensor, and in particular, the wedge membrane 195 are in engagement with at least one, and preferably a plurality of antennas 201 formed within the interior wall surface 225 of the detection means.

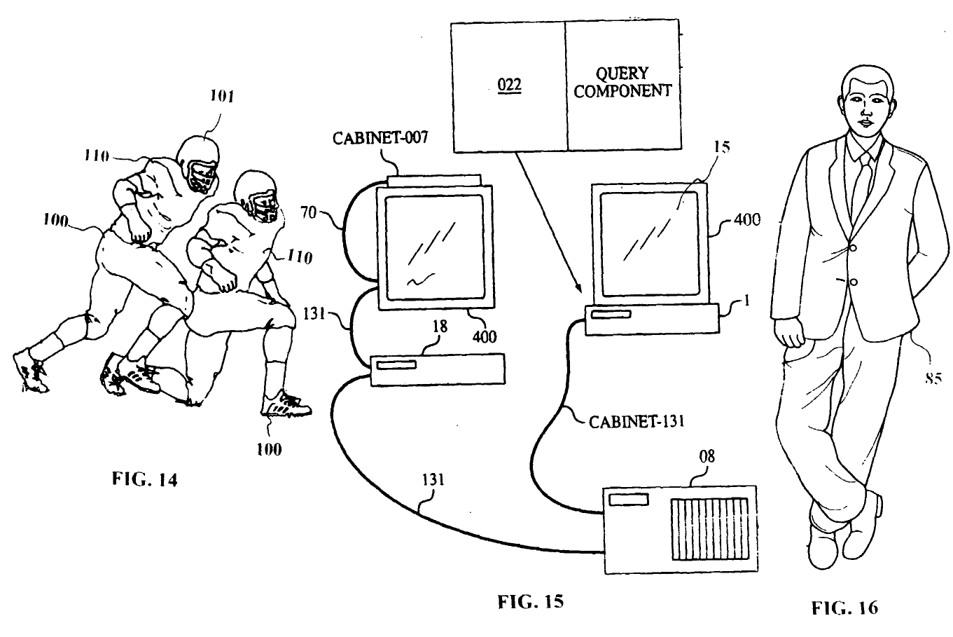

The present invention relates to a technology for providing comprehensive analytical logistics to on-field applications to physiological change through a wearable detection platform. The platform allows various teams to research on on-field conditions of players information by retrieving the information from the outfit to a computer device 400 located at proximity to the field. In other embodiment, the computer device is a desktop computer 1, a network computer such as server 00. FIG. 14 depicts players 110 on the sideline responding to transmitted on-field communication to a communication device 400 shown in FIG. 15. The communication device comprises at least a memory 022 configured with a query component, a cabinet 007 configured with a screen device 15, and a computer means 18. Wire harnesses 70, 131 are responsive for transmitting signals. At least a PDC 08 could be configured to store and communicate all aspects of the detections to at least another computer device, such as the server 00.

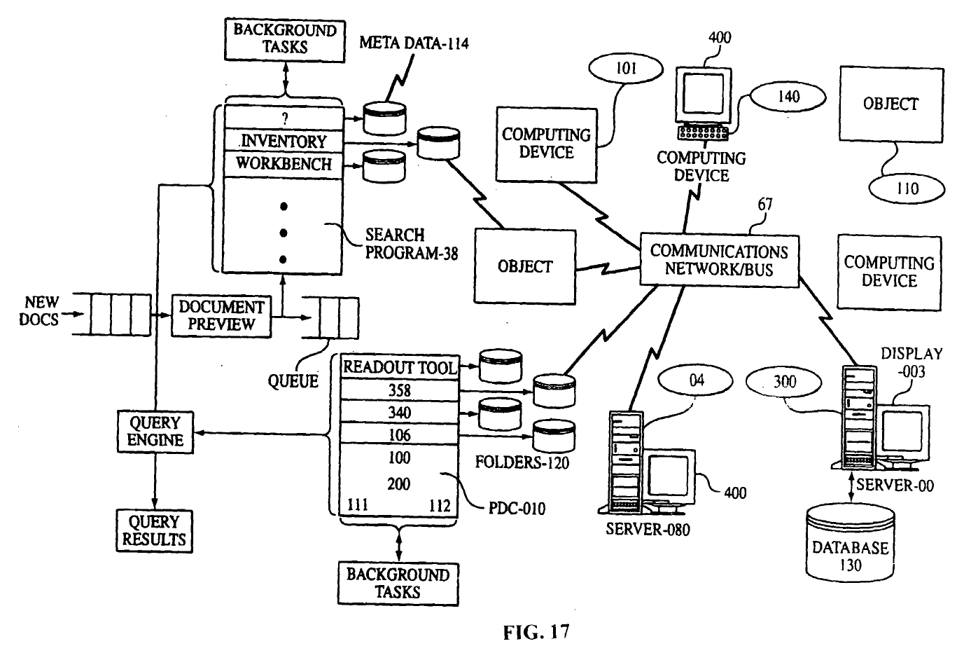

FIG. 16 depicts personnel 85 of a football team watching the communications transmitted from the field to the computer device 400. FIG. 17 depicts aspects of the present invention comprising a centralized network, such as NFL NETWORK comprising servers 080, 00, and configured with at least a PDC 010. The PDC 010 comprises read out tools 358, 340, 112, 111 106, 100. The readout tools represent different parts of the physiological logistical change, and are communicatively configured with folders 120, in communication with a communication network 67. The communication network 67 could be distributive, enabling communications to at least a computer device.

The server 00 is configured to enable communication with hand held computers, wireless devices, and laptop computers. The server is 00 is operatively configured with software 300. The software 300 functions as the operating system for computer 400, server 00, 080, PDC 010 and the applications that enable communication with other devices and computers. The software 300 allows the server tools to communicate with each other while the application permits multiple devices to perform search task. The tools enable communication with the ROM, while allowing the software 300 to communicate with the hardware of the server computer 00, enabling security and reliability on the team files 114 and folders 120. The team files 114 or folders 120 are created in another database 130 through a primary domain controller “PDC” 010, or another set of file/folder server for keeping statistical teams data.

The PDC 010 centralizes all team computers through network adapters or wireless communication device. The network adapters provide the physical connection to the network, locating the physical addresses of team computers or other devices. The PDC 010 receives the team, and/or password, at the initial logon screen, and creates security identification that will set the permission and rights for the team services. Connectivity is allowed through protocol communication, permitting data to be sent through network adapters into cables 131 or other wireless means. The network interface pulls out or put into network adapters, the data through the cables 131 with no internal protocol. The communication process from one computer to a network, to retrieving data, passes through model layers that are assigned specific task to the layers.

The background tasks are operatively connected to an inventory workbench, which is configured with search program 38. The workbench is communicatively connected to at least a document preview in communication with at least a query engine. The query engine comprises query results communicatively connected to the background tasks and the search program is operatively connected to object folders 110. In other embodiment, computer device 101 comprises at least a key board 140 and at least a display 003. Database folder 130 is communicatively connected to server 00 operatively connected to communications network/bus. In still another embodiment of the present invention, server 080, 00, and PDC may storage means 04.

It is to be understood that the scope of the present invention is not limited to the above description, but encompasses the following claims;

What is claimed:

1. Advanced force responsive outfit configured for enabling detection and for classifying physiological conditions of personnel; comprising:
a. at least a sensor means;
b. said sensor means comprises at least a detection means comprising a platform responsive to at least a detection; and
c. at least a communication means.

2. Advanced force responsive outfit of claim 1, wherein said sensor means comprises at least a nano-sensor means.

3. Advanced force responsive outfit of claim 1, wherein said sensor means operatively configured to enable at least a prescribed detection.

4. Advanced force responsive outfit of claim 3, wherein said detection means comprises at least a detection platform further responsive to detection and for monitoring on-field logistics to physiological change.

5. Advanced force responsive outfit of claim 4, wherein said detection platform is at least force responsive and further comprises at least a nanotechnology application.

6. Advanced force responsive outfit of claim 1, wherein said detection means communicatively connected to at least a communication means.

7. Advanced force responsive outfit of claim 1, wherein said sensor means further comprises at least a pressure sensitive sensor means.

8. Advanced force responsive outfit of claim 7, wherein said pressure sensitive sensor means further comprises means for transforming force energy into at least electrical energy.

9. Advanced force responsive outfit of claim 1, wherein said detection means further comprises means for detecting collision force.

10. Advanced force responsive outfit of claim 1, wherein said detection means further comprises means for monitoring body logistics, said body logistics further comprises at least a condition to physiological change.

11. Advanced force responsive outfit of claim 10, wherein said body logistics further include at least a characteristic influential to least a change in physiological conditions.

12. Advanced force responsive outfit of claim 8, wherein said electrical energy further comprises at least a measurable force.

13. Advanced force responsive outfit of claim 1, wherein said detection means operatively configured with said communication means, said communication means further responsive to at least a measurable force communications.

14. Advanced force responsive outfit of claim 13, wherein said communication means further comprising means for communicating said measurable force.

15. Advanced force responsive outfit of claim 1, wherein said communication means further comprising means for communicating at least a change in physiological conditions.

16. Advanced force responsive outfit of claim 1, wherein said communication means operatively configured to communicate with at least a remote communication means.

17. Advanced force responsive outfit of claim 16, wherein said remote communication means comprising at least a computer means.

18. Advanced force responsive outfit of claim 17, wherein at least said one computer means positioned at least at vicinity proximately within an environment of at least a sporting event.

19. Advanced force responsive outfit of claim 17, wherein said computer means further comprises at least a server means.

20. Advanced force responsive outfit of claim 19, wherein said server means comprising means for communicating to at least said one remote communication means.
